(19) United States
(12) Patent Application Publication
(10) Pub. No.: US 2026/0176225 A1
(43) Pub. Date: Jun. 25, 2026

(54) Novel Catalysts for Highly Selective Formation of Oxygenates from Syngas

(71) Applicant: University of Cincinnati, Cincinnati, OH (US)

(72) Inventors: Thirupathi Boningari, Cincinnati, OH (US); Panagiotis G Smirniotis, Cincinnati, OH (US)

(73) Assignee: University of Cincinnati, Cincinnati, OH (US)

(21) Appl. No.: 19/430,166

(22) Filed: Dec. 22, 2025

Related U.S. Application Data
(60) Provisional application No. 63/737,594, filed on Dec. 20, 2024.

(51) Int. Cl. C07C 29/17; C07C 31/08

(52) U.S. Cl. CPC C07C 29/172; C07C 31/08

(57) ABSTRACT

A process for producing oxygenates from syngas is provided. The process involves mixing syngas with hydrogen sulfide, forming a syngas mixture; contacting the syngas mixture with a catalyst composition in a reactor to obtain a first product stream; splitting the first product stream into a non-condensable material stream and a condensable material stream; directing the non-condensable material stream back into the reactor; and directing the condensable material stream to a distillation column, producing a product stream having a high percentage of ethanol. The catalyst composition includes rhodium, lanthanum, gallium, vanadium and a support.

### CROSS-REFERENCE TO RELATED APPLICATIONS

This application claims priority to, and the benefit of the filing date of, U.S. Provisional Application No. 63/737,594 filed Dec. 20, 2024, the disclosure of which is incorporated by reference herein in its entirety.

### FIELD OF THE INVENTION

This invention relates generally to a process for converting syngas.

### BACKGROUND OF THE INVENTION

Increasing concerns about global climate change, depletion of fossil fuel resources, and rising crude oil prices have pushed the topic of energy to the center stage. Bio-based fuel resources, particularly ethanol, have been studied extensively in recent years as clean, sustainable and transportable fuel alternatives. In fact, over 2 billion gallons of ethanol were produced in the US in 2002, mostly for use as a fuel additive. Although this is small fraction of the US consumption of 134 billion gallons per year of gasoline, studies show that there is a potential to increase ethanol production to 34-75 billion gallons per year. As a fuel, ethanol has several ideal properties: it is nontoxic, easy to store and also ethanol transportation fuel results in lower net petroleum use and lower greenhouse gas emissions than gasoline per mile driven. In addition to its potential application as a transportation fuel, bioethanol has been considered as a feedstock for the synthesis of variety of chemicals, fuels, and polymers. Consequently, there is a growing worldwide interest in the production of ethanol from biomass and possibly from other readily available carbonaceous sources such as coal without CO2 emission, and its use as a fuel for transportation, chemical feed stocks, and as an H2 carrier in the future.

Currently, ethanol is produced by two major processes: (1) fermentation of sugars derived from corn or sugar cane and (2) hydration of petroleum-based ethylene. The ethylene hydration route is unattractive for large-scale production of ethanol because of rising crude oil prices and the dependence on imported oil. Although the fermentation route is commercially practiced for the production of most of the ethanol produced today, the production of fuel-grade ethanol is expensive and energy-inefficient because the process involves energy intensive distillation steps. Production of ethanol-using thermochemical route i. e. conversion of syngas to ethanol is the alternative route and received much attention in recent days because syngas can be conveniently manufactured from natural gas, coal and biomass. However, the catalytic conversion of syngas to ethanol remains challenging and no commercial process exists as of today although research on this topic has been ongoing for 90 years. One of the major obstacles of this process is low selectivity of ethanol.

The catalysts for the synthesis of ethanol from syngas can be classified into four categories: (a) Rh based catalysts (b) modified methanol synthesis catalysts (c) modified Fisher-Tropsch catalysts (d) Modified Mo-based catalysts. Among the various catalysts, Rh-based catalysts have the highest selectivity for C2+ oxygenated compounds such as ethanol. The most relevant feature of Rh is its ability to adsorb reactive CO both associatively and dissociatively, allowing it to form both hydrocarbons and oxygenates. Because Rh is an expensive metal, the improvement of activity and ethanol selectivity over Rh-based catalysts is necessary for achieving a commercially available process. The influence of various promoters on the catalytic activity of Rh has been extensively studied in recent years. It is proposed that the carbon atom of a CO molecule binds to the Rh atom and the oxygen atom binds to a neighboring promoter cation which can either weaken the C—O bond resulting in CO dissociation or promote CO insertion.

Most previous work has been performed over SiO₂-supported systems. Examples of other supports studied include Al₂O₃, TiO₂, NaY, ZrO₂ and CeO₂. Mesoporous molecular sieves like SBA-15, MCM-41, MCM-48, and MCF have received much attention for potential applications as versatile catalysts and supports because of its appealing textural properties and high surface area, and appreciable thermal and hydro thermal stability. Extensive research efforts have been devoted to study reaction mechanisms and the effects of supports and promoters on Rh-based catalysts using various characterization techniques. Infra-red spectra (IR) has been employed widely for studying the Rh-based catalysts and examples of other characterization techniques include temperature programmed reduction (TPR), hydrogen chemisorptions, X-ray photo electron spectroscopy (XPS), etc. While the basic features of the mechanism are clear, the actual mechanisms are unclear over these catalysts. Therefore, the catalytic and selective conversion of syngas to ethanol and oxygenates remains challenging and no commercial process exists as of today with one of the major obstacles of this process being the low selectivity for the desired products.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, a process for producing oxygenates from syngas is provided. The process involves the steps of a) mixing syngas with hydrogen sulfide, forming a syngas mixture; b) contacting the syngas mixture with a catalyst composition in a first reactor to obtain a first product stream including ethanol, water, hydrogen, carbon monoxide and hydrogen sulfide; c) splitting the first product stream into a non-condensable material stream including hydrogen, carbon monoxide and hydrogen sulfide and a condensable material stream comprising water and ethanol; d) directing the non-condensable material stream back into the first reactor; and e) directing the condensable material stream to a first distillation column, producing a second product stream having a majority of water and a third product stream having a majority of ethanol. Further, the catalyst composition includes rhodium, lanthanum, gallium, vanadium and a support.

In one embodiment, the support for the catalyst composition is selected from the group comprising SiO₂, zeolites, silicalites, mesoporous silicas, alumina, aluminophosphates, activated carbons, and carbon nanotubes. In another embodiment, the support for the catalyst composition is SiO₂. In one embodiment, the catalyst composition comprises 1.5Rh-1.5La-1Ga/1.8V/SiO₂.

In another embodiment, the hydrogen sulfide is present in the syngas mixture at a level of 400 ppm or greater. In one embodiment, the hydrogen sulfide is present in the syngas mixture at a level of about 400 ppm.

In one embodiment, the third product stream comprises at least about 90 weight percent ethanol. In another embodiment, the third product stream comprises at least about 95 weight percent ethanol.

In another embodiment, the third product stream is directed to a second distillation column, producing a fourth product stream comprising a majority of water and a fifth product stream comprising a majority of ethanol. In one embodiment, the fifth product stream comprises at least about 90 weight percent ethanol. In another embodiment, the fifth product stream comprises at least about 95 weight percent ethanol.

In one embodiment, the syngas has a hydrogen (H₂) to carbon monoxide (CO) molar ratio of about 1-4. In another embodiment, the syngas has a hydrogen (H₂) to carbon monoxide (CO) molar ratio of about 2.

In one embodiment, the first reactor is operated at a temperature from about 150° C. to about 350° C., a space velocity of about 5000-7000 sccm gcat-1h-1 and a pressure of between atmospheric and about 700 psig. In another embodiment, the first reactor is operated at a temperature of about 260° C. In one embodiment, the first reactor is operated at a pressure of about 350 psig. In another embodiment, the first reactor is operated at a space velocity of about 6000 sccm gcat-1h-1. In one embodiment, the first product stream is split into a non-condensable material stream and a condensable material stream using a heat exchanger.

In another embodiment, the syngas is sourced from coal. In one embodiment, the syngas is treated to remove acid gases using a physical solvent that is a mixture of dimethyl ethers of polyethylene glycols prior to mixing the syngas with hydrogen sulfide.

### BRIEF DESCRIPTION OF THE DRAWINGS

The objects and advantages of the disclosed invention will be further appreciated in light of the following detailed descriptions and drawings in which:

FIG. 1 is a schematic showing the integrated process to transform coals to syngas via a chemical looping process and the transformation of syngas to oxygenates.

FIG. 2 is a schematic showing the process to transform coals to syngas via the chemical looping process.

FIG. 3 is a schematic showing the effect of H₂S on the CO conversion and selectivity towards oxygenates and methane in the syngas to oxygenates reaction over Rh—La—Ga/V/SiO₂ at 260° C. for 31 days; Reaction conditions: H₂S=400 ppm, P=350 psig, H₂/CO=2, S.V.=6,000 sccm gcat⁻¹h⁻¹.

FIG. 4 is a schematic showing the effect of H₂S on the selectivity of various oxygenates in the syngas to oxygenates reaction over Rh—La—Ga/V/SiO2 at 260° C. for 31 days; Reaction conditions: H₂S=400 ppm, P=350 psig, H2/CO=2, S.V.=6,000 sccm gcat-1h-1. The detailed product distribution as a function of time is shown in Table 18.

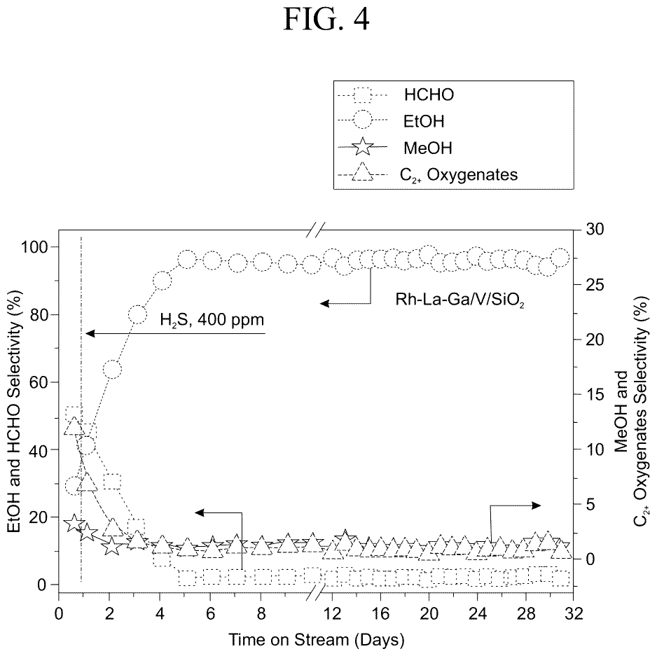

FIG. 5 is a graph showing the effect of H₂S on the CO conversion in the syngas to oxygenates reaction over Rh—La—Ga/V/SiO₂ at 260° C. for 4 days; Reaction conditions: H₂S=100 ppm, P=350 psig, H₂/CO=2, S.V.=6,000 sccm gcat⁻¹h⁻¹.

FIG. 6 is a schematic showing the effect of H₂S on the selectivity towards oxygenates and methane in the syngas to oxygenates reaction over Rh—La—Ga/V/SiO₂ at 260° C. for 4 days; Reaction conditions: H₂S=100 ppm, P=350 psig, H₂/CO=2, S.V.=6,000 sccm gcat⁻¹h⁻¹.

FIG. 7 is a graph showing the effect of H₂S on the CO conversion in the syngas to oxygenates reaction over Rh—La—Ga/V/SiO₂ at 260° C. for 4 days; Reaction conditions: H₂S=1,000 ppm, P=350 psig, H₂/CO=2, S.V.=6,000 sccm gcat⁻¹h⁻¹.

FIG. 8 is a schematic showing the effect of H₂S on the selectivity towards oxygenates and methane in the syngas to oxygenates reaction over Rh—La—Ga/V/SiO₂ at 260° C. for 4 days; Reaction conditions: H₂S=1,000 ppm, P=350 psig, H₂/CO=2, S.V.=6,000 sccm gcat⁻¹h⁻¹.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In one embodiment, the present invention involves a highly selective process for producing ethanol from syngas. The syngas can come from any source. These sources include coal, natural gas, petroleum products, biomass, agricultural waste, and captured CO₂. The process involves mixing syngas with a low level of hydrogen sulfide and reacting with a catalyst composition comprising rhodium, lanthanum, gallium, vanadium and a support. The support may be SiO2, TiO₂, NaY, ZrO₂, CeO₂, mesoporous molecular sieves like SBA-15, MCM-41, MCM-48, and MCF, zeolites (including ZSM-5, SSZ-13, and Y-type (FAU), silicalites, mesoporous silicas (such as MCM-41 and KIT-6), alumina, aluminophosphates, activated carbons, and carbon nanotubes. In one embodiment, the support is SiO₂. In another embodiment, the catalyst is Rh—La—Ga/V/SiO₂. The resulting product stream is split, with the condensable material being distilled to produce a high percentage of ethanol.

One particular example involves the production of syngas by using coal with a chemical looping process. In this embodiment, an integrated process for transforming coals into ethanol comprises chemical looping to transform coals into syngas with an H₂/CO ratio of 2. The thus generated syngas is supplied to a reactor to produce almost exclusively EtOH while the unreacted syngas is recycled back to the reactor to further transformed into ethanol.

The mode of operation at the selected operating conditions of the main reactors basically transforms all syngas into ethanol. The acid gases that are produced from the chemical looping process and the small amount of H₂S that comes out of the main reactor (H₂S is fed into the main reactor to ensure stability and promote the almost exclusive production of EtOH) is fed to a physical solvent process for purification. The sulfur is also transformed into H₂SO₄ that adds to the total revenue of the process. The process can operate 24 hours a day year-round, excluding holidays and scheduled and unscheduled downtime, for about 8,000 operating hours a year. Coal can be fed into the process at 3,000 tons per day.

The coal utilized in the data presented herein is Ohio bituminous-grade coal, which is notably high in sulfur content. In one design shown herein, the coal can be fed into the process at 3,000 tons/day into a grinding process. Once the coal has been ground to 100 mesh, the coal is fed into the first reactor along with steam and an Fe₂O₃ oxygen carrier catalyst. Multiple alternate fuels were investigated as supplemental fuel sources as the process runs endothermically overall, and coal was chosen as the heat source. In this reduction reaction, syngas product can be produced along with the byproducts fly ash and solid FeO catalyst. The solid FeO is then fed into a second reactor, where a combustion process in air produces solid Fe₂O₃ catalyst. The catalyst is separated from the oxygen depleted air, with the spent air having less than 10% CO₂.

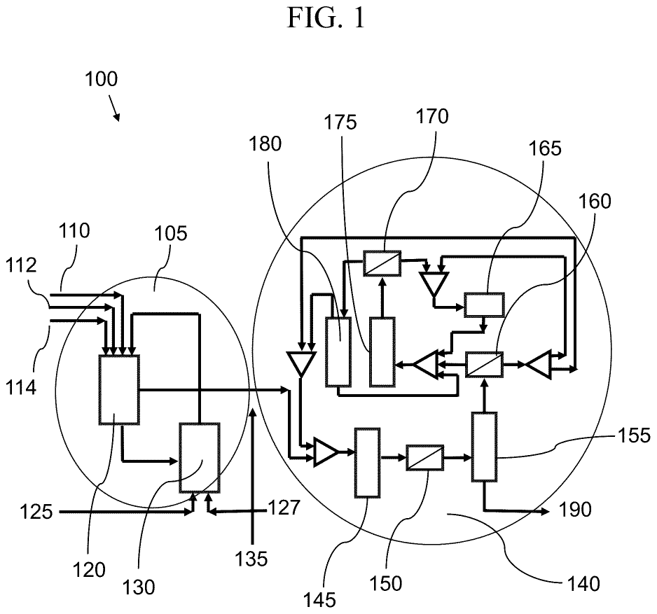

Referring to FIG. 1, an integrated system 100 includes a chemical looping portion 105 and a syngas to oxygenates portion 140. For the chemical looping portion 105, natural gas 110, steam 112 and coal 114 enter a reducer 120 at about 10 bar. Additionally, coal 125 and air 127 enter a combuster 130. The output of the combuster 130 is reacted with a Fe₂O₃ catalyst and directed into the reducer 120. Syngas produced in the reducer 120 goes to the syngas to oxygenates portion 140, while the remaining output from the reducer 120 goes back to the combuster 130. Selexol 135 is introduced.

The syngas to oxygenates portion 140 includes: an OXY reactor 145, an H₂S separator 150, an OXY Flash 155, which outputs oxygenates 190, with the remainder being sent to an H₂ separator 160. Further, the syngas to oxygenates portion 140 includes an RWGS 165, a CO₂ membrane separator 170, a reformer 175, and an H₂O Flash 180.

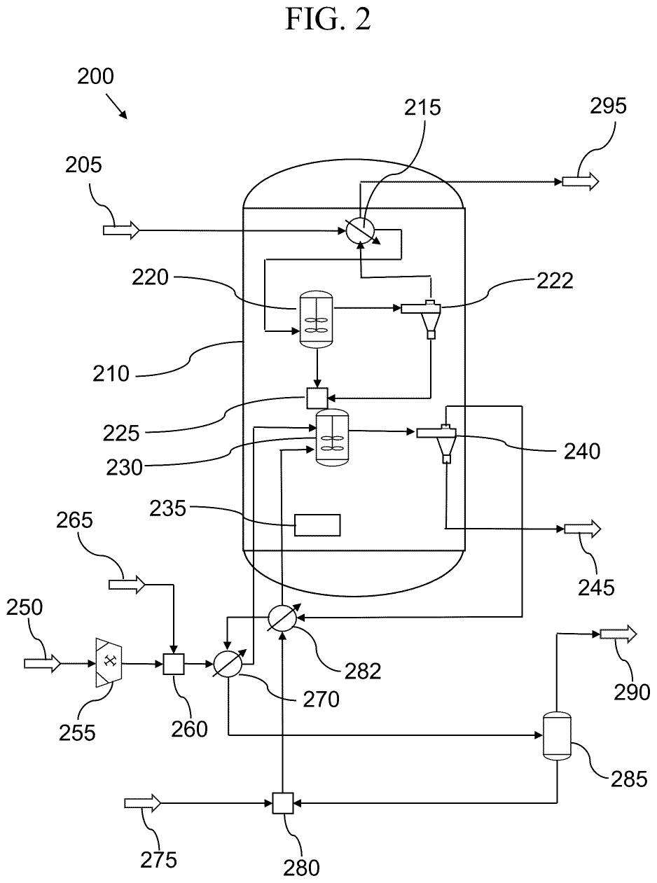

A chemical looping process 200 is shown in FIG. 2. Air 205 enters a V-225 reaction pressure vessel 210. The V-225 reaction pressure vessel 210 includes: an E-120 air exchanger 215, R-250 combustion reactor 220 and refractory brick 225, F-310 Fe₂O₃ cyclone 222, an R-200 reduction reactor 230, an H-205 reaction heater 235, and an F-300 fly ash cyclone 240, from which fly ash and deactivated catalyst 245 exit. Depleted air 295 exits from the E-120 air exchanger 215.

A coal feed 250 sends raw coal to a U-100 coal grinder 255, producing 100 mesh coal and directing it to an exchanger 260. Fresh Fe₂O₃ is also supplied to the exchanger 260. The output of the exchanger 260 is sent to an E-110 coal preheater 270. Process water 275 is sent to a second exchanger 280 as well as water recycle from a V-340 water decanter 285. The second exchanger 280 sends its output to an E-320 water condenser 282. Dry syngas product 290 is output from the V-340 water decanter 285.

### Chemical Looping

In one aspect of the present invention, syngas is produced from coal using a chemical looping process, which can later be processed into ethanol, or another oxygenate product. The process can operate 24 hours a day year-round, excluding holidays and scheduled and unscheduled downtime, for about 8,000 operating hours a year. Coal can be fed into the process at 3,000 tons per day, or about 125 pounds per hour. Recycled Fe₂O₃ can be fed into the process at 10 tons per hour, and steam can be fed into the process at 100-130 tons per hour. The expected production rate of dry syngas is 240-280 tons per hour.

The concept of chemical looping is a new concept that has been researched at the Ohio State University. One of the goals of this process design is to eliminate the feed of natural gas and substitute the feed with an alternate fuel source that would be the most economical. It should be noted that OSU used PRB coal which is lower in sulfur content than Ohio bituminous coal.

For testing the present invention, coal compositions were gathered from Belmont and Caroll County in Southeast OH and the minimum and maximum weight percentages were recorded. The Belmont coal sample is a useful coal for this process; however, coal varies in composition within counties, so we decided to design for a worst- and best-case scenario, provided the syngas product will have a 2:1 hydrogen/carbon monoxide ratio. Incoming coal is roughly the 30 mm diameter size fraction. Analytical methods are used to ensure the coal entering the process falls within the ranges in Table 1; any coal out of speculation will be burned as a heat source.

Analytical methods are used to qualify or characterize the incoming coal feed to ensure the correct compositions that the process calls for in the recommended design. Sampling techniques for solid samples will need to be researched in order to ensure that the sub-samples collected represent the bulk stream of coal. An example of a sampling method that can be used would be the Automated Reference Method Sampler. This method sampler would attach to a conveyor belt and would have minimal process interruptions. More sampling analysis may be needed later in the process. When the coal is within the parameters laid out in Table 1, the process controls will be adjusted to ensure quality syngas, whereas coal that does not meet the feed requirements will be burned for heat, so no coal is wasted. For the downstream oxygenate formation to work, the syngas needs to have a H2:CO ratio of 2:1, so the incoming coal composition is important for process control.

The oxygen carrier catalyst is prepared offsite from a 50:50 weight fraction iron oxide catalyst on a silicon dioxide support, produced at a particle size of 1.5 mm. Process water is pulled from the Ohio River and purified at the onsite water treatment plant. Atmospheric air will be used to supply the oxygen to R-250.

The flowrates of the feed streams entering the reactor, R-200, are shown in Table 2. The approximate composition of the syngas product is shown in Table 4. The syngas composition can be determined by UV-vis spectrometry with a gas chromatograph. The H₂S is an undesirable component of syngas, and it can be treated after the syngas purification process.

The byproduct stream flowrates are shown in Table 5. Fly ash is composed mainly of a mixture of iron oxides, silicon oxides, magnesium oxides, and aluminum oxides, the ratio of which depends heavily on the specific coal. The fly ash and deactivated catalyst will be sold for concrete production. More research can be done to determine if the deactivated catalyst could be separated for profit and the appropriate analytical method to test the quality of the final product will need to be researched as well. Spent air is assumed to have half of its original oxygen content at 10.5%, and will be discharged to the atmosphere after being cooled to 90° F.

### Coal Processing

Heat will be required to preheat the feed streams, with most of it provided by the heat integration and the rest supplied by burning coal. The reduction reactor runs endothermically, and the combustion reactor runs exothermically at between 410 and 1,870 MMBtu/hr and between −620 and −860 MMBtu/hr respectively. If 95% of the heat generated by the combustion reactor can be recycled to the reduction reactor, the additional heater will only need to provide between −180 (exothermic) and 1,050 MMBtu/hr to sustain the chemical reactions. It was assumed that the variation in coal would allow an average of these two values to be taken when sizing the heater. All other energy requirements will also be supplied by H-205, bringing the total energy requirement for H-205 up to 601 MMBtu/hr. Heat for preheating air into the combustion reactor can be supplied by the spent air leaving the reactor via E-120. The coal to fire H-205 and electricity to power U-100 and the conveyers are therefore the only utilities required for this process. Fly ash disposal can be sold to concrete companies for concrete production. Excess sulfur will be cleaned and handled by another project group downstream. Wastewater will need to be treated on-site.

As an example, a proposed facility location is Southeast Ohio. Ohio bituminous coal can be used, therefore, a facility location in Ohio would cut down on transportation costs. The facility will need to be near a river or a large aquifer because a lot of water is needed for the process. The most economical way to transport coal would be by barge compared to trucking and railroad as shown in Table 7.

In one useful design, there are three process areas. A coal prepping area, a reactor area and a separation unit area. In future work, there is a recommendation for an additional area that would include raw material and product storage. These three areas will have a minimum of 100 ft between them to protect against dust explosions or chain reaction failures.

### Chemical Looping Process

In one embodiment, the process of the present invention conducts circular looping of an oxygen carrying catalyst through an oxygen deficient fluidized bed reactor along with coal dust. The catalyst is reduced in this reaction with coal, as shown in FIG. 2. This is done in the presence of steam to optimize the production of H₂ and CO instead of the thermodynamically favorable CO₂. The reduced FeO catalyst is then oxidized by burning in air in a second fluidized bed reactor.

Coal ground to 100 mesh (150 micron), oxygen carrying catalyst (active Fe₂O₃ on SiO₂ support) at 1.5 mm particle size, and steam is fed into reactor R-200. The carbon in the coal is oxidized by the Fe₂O₃ to CO and CO₂ to form syngas. This reaction takes place at 1590° F. and 10 bar. The reduced FeO is fed into the second reactor, R-250, and reacted with air to oxidize back to Fe₂O₃. The air will be depleted to about 50% of its original oxygen concentration in the reaction. In one embodiment, this reaction takes place at 2160° F. and 10 bar. The Fe₂O₃ is then recycled back into the R-200 reactor. The catalyst should be able to maintain efficacy at up to 100 cycles based on the research done by OSU. However, they did not test it to failure so it is possible that the catalyst could retain its potency for longer, which would reduce the raw catalyst and deactivated catalyst streams.

In one embodiment, a fluidized bed is used for the mixing of the different solid particles and heat transfer between drastically different temperature streams. It was also realized that the temperature of R-250 is incompatible with all stainless steels, since these metals would suffer unacceptable levels of creep over the lifetime of the project. Other metal alloys were investigated, but the only candidates, molybdenum and tungsten alloys, were prohibitively expensive. It was then theorized that ceramics could be used due to their exceptional temperature tolerance and lack of vulnerability to creep. Refractory brick was selected due to its low cost and density to be the bulk of the reactors' structure, and this was determined using GRANTA CES EduPack. However, refractory brick has a very low fracture toughness and would abrade over time from contact with the fluidized bed. To prevent this, an alumina liner was proposed. This liner would be much thinner and made from aluminum oxide, which has a much higher fracture toughness.

### Description of the Syngas Purification Design

In one embodiment, the present invention involves a process to remove acid gases from two hydrocarbons streams. One of these streams is syngas generated from coal and contains a large amount of CO₂ and H₂S while the other stream is a reactor effluent stream that contains no CO₂ and a small amount of H₂S. The removed acid gases can be used to create other usable products. A physical solvent that is a mixture of dimethyl ethers of polyethylene glycols was chosen for the process as it provides better removal for high CO₂ conditions than other solvent options. An example of such a solvent is “Selexol,” available from the Dow Company. Recovered CO₂ can be used as is for other industrial applications, while H₂S is sent to a Wet Sulfuric Acid (WSA) process unit to be converted into liquid sulfuric acid.

For the syngas purification process, there will be a waste stream separated from the main acid gas stream that is sent to the WSA unit. This waste stream contains mostly water and methanol, with small amounts of other contaminants. Additionally, there is a waste stream from the WSA scrubber tower scrubber that contains mostly water and calcium carbonate, with small amounts of other contaminants. These wastewater streams will be sent to a treatment facility before being discharged to the sewer.

The syngas purification unit contains two primary feed streams for each treatment location. The first is syngas from the chemical looping process received in the amount of 247.2 tons/hr, and Selexol solvent which circulates at a rate of 500 tons/hr. The oxygenates effluent Selexol treating unit receives its feed stream in the amount of 9.14 tons/hr, and circulates 175 tons/hr of the Selexol solvent.

The wet sulfuric acid process receives the isolated H₂S from the two Selexol units for a total gas flow rate of 80.51 tons/hr. Additionally, 77.35 tons/hr of ambient air is mixed into the gas feed for the combustion process. The flow rate of the lime solution to the SO₂ scrubber is 5.99 tons/hr. All processes are expected to operate for 8,000 hours per year.

### Feed Specifications

Feed specifications are displayed in Tables 11-13 below. Feedstock information for the two Selexol units have been supplied by the Chemical Looping of coals and the H₂S leaving the reactor that transforms Syngas to Oxygenates, while the wet sulfuric acid process is drawing feedstock information from the two Selexol units.

### Purification Process Description

This process removes undesired H₂S and CO₂ from two different gas streams. One feed stream is syngas generated from Ohio-based coal, and the other is an effluent stream from a reactor that converts the syngas into oxygenate products. The latter stream contains a trace amount of H₂S, while the syngas feed is rich with both acid gases. The recovered CO₂ can be sold as is to industries needing the gas in lower purities or further purified to be sold as more specialized product. The H₂S removed from the feed gas is routed to a Wet Sulfuric Acid (WSA) unit to be converted into high purity sulfuric acid. This conversion to sulfuric acid is accomplished with the series of reactions below:

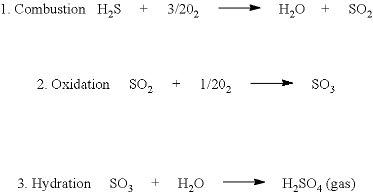

A hydrogen sulfide conversion of 90% was targeted for the WSA process with reactor simulations being carried out on a stoichiometric basis. These reactions are assisted by the Topsoe VK series of catalysts composed of vanadium pentoxide and sodium/potassium pyrosulfates. The highest performing vanadium pentoxide catalysts are those that have the geometric structure of nanowires. The Topsoe VK catalysts will be packed in a series of single bed stoichiometric reactors to convert sulfur dioxide to sulfur trioxide. The beds in each reactor (from bottom to top) are composed of a carbon steel grate, layered with earth silica packing support media and topped with the vanadium pentoxide catalyst.

Three options for the removal of the acid gases and two options for the further treatment of recovered H₂S were considered before the recommended process was decided upon. Acid gases can be extracted by using methyl diethanolamine (MDEA) as a chemical solvent or by using one of two physical solvents: Rectisol or Selexol. Ultimately, MDEA and Rectisol were eliminated as possibilities due to the composition of the syngas feed requiring treatment. The syngas feed contains a high enough amount of CO₂ that it is no longer within the ideal ranges for MIDEA or Rectisol systems. Selexol, however, is suited well not only for the higher load of both acid gases but for systems with more CO₂ than H₂S.

The process included in this part of the project consists of three separate process units: The syngas Selexol treating unit, the oxygenate effluent Selexol treating unit, and the wet sulfuric acid process unit.

Syngas produced from coal via the chemical looping is sent to a first solvent unit containing a physical solvent that is a mixture of dimethyl ethers of polyethylene glycols, such as Selexol, to remove the CO₂ and H₂S. This is accomplished with the physical solvent which contacts the syngas in an absorber to first remove the H₂S. The overhead gas is sent to another absorber to remove the CO₂ while the solvent containing the acid gas is sent to a regenerator tower. The regenerator tower, and its preflash equipment, separates the solvent from the absorbed gas and sends the recovered gas on to further treating while recycling the solvent. The second absorber tower functions very similarly to the first, and is also followed by equipment to separate the rich Selexol from its dissolved acid gases.

The second Selexol process treats oxygenate product stream from the Syngas to Oxygenates reactor/project that is synthesized from the treated syngas produced by the Chemical Looping of coals project. The layout of this second Selexol unit is similar to the first half of the other Selexol process, however the oxygenate treating does not require the removal of CO₂ and is removing a much smaller load of H₂S.

Removed H₂S from the Selexol processes is routed to a Wet Sulfuric Acid (WSA) process unit to be converted into marketable sulfuric acid. The WSA process combines the recovered H₂S with preheated air and combusts the mixture to produce sulfur dioxide. The resulting mixture of sulfur compounds is sent through a reactor train to first convert the sulfur dioxide into sulfur trioxide and then utilize the water created through the combustion process to convert the sulfur trioxide to sulfuric acid. This reactor train is aided by the presence of a vanadium (V) oxide catalyst, which typically requires replacement or regeneration after six years.

The combustion of the H₂S is not 100%, and there is unreacted hydrogen sulfide that goes through the rest of the wet sulfuric acid process. After the sulfuric acid condenser, the tail gas goes through a sulfur dioxide scrubber for unreacted sulfur dioxide. The remaining gas then goes through a wet-type electrostatic precipitator to remove a majority of the unreacted sulfur trioxide. This leaves the final gas stream with unreacted hydrogen sulfide, oxygen, and nitrogen. Unless the conversion of the combustion reactor can be increased, the unreacted hydrogen sulfide must be treated.

The two Selexol processes mostly feature separation equipment in addition to heat exchangers, pumps, and compressors while the WSA process contains all of the reaction equipment for our recommended process. Key pieces of equipment are displayed in Table 14 below.

### Oxygenate Synthesis Process

One embodiment of the present invention is the synthesis of oxygenates (methanol, ethanol, and propanol) from syngas (hydrogen and carbon monoxide). The primary oxygenate produced in this process is ethanol. The side products from the reactions are water and formaldehyde. While the syngas source used in the examples is coal, other sources for the syngas can be used. These sources include natural gas, petroleum products, biomass, agricultural waste, and captured CO₂.

In one embodiment, the primary chemical reaction of this process is reacting syngas composed of hydrogen and carbon monoxide in the ratio of 2:1 delivered from chemical looping of coals and creating oxygenates or alcohols from the reaction. Previous attempts at this process revealed some unwanted side reactions that produced methane. The production of methane made the separation process extremely difficult. It was then discovered that the addition of a small amount of hydrogen sulfide (400 ppm) prevented the production of methane. The reactor was also operating at a relatively low temperature (for example, 260° C.) to prevent the production of another unwanted side product, carbon dioxide. The reaction primarily produces ethanol and water with small amounts of methanol, propanol, and formaldehyde.

The oxygenates, water, and leftover reactants then flow to a heat exchanger where the non-condensables of the system (carbon monoxide, hydrogen, and hydrogen sulfide) are recycled back into the reactor, while the remaining products are sent to the separations section of the process. Since the reaction is not an exact 2:1 ratio of hydrogen to carbon monoxide, a small purge stream of mostly hydrogen is vented from the recycle stream and sent back to the front of the process.

In one embodiment, the separations section is two distillation columns. The first column separated mostly pure water out of the bottom of the column and a 96% pure ethanol stream with some water and other trace side products such as methanol. The second distillation column further purified the ethanol to 98 wt % and sent a small stream of mostly methanol, propanol, and water to a mixer where the water stream from the first column mixed together.

An alternative design is to have a far more complex separation process to capture the trace oxygenates of methanol, formaldehyde, and propanol that were produced in the reaction. Since the flow rates of these other products are extremely low, the costs of additional separation equipment to obtain these products in pure form were not determined to be economically viable.

### Distillation Columns

In one embodiment, the distillation columns in this plant are used after the stoichiometric reactor to separate ethanol from water and the trace oxygenates. The number of distillation columns used is 2, but more distillation columns could be used. In alternative embodiments, 3 or 4 distillation columns could be used.

### Boiling Points, Volatility, Reflux Ratio, and the Feed Point Location

The separation of liquid mixtures by distillation depends on the differences in volatility between the components. The greater the difference between the components, the easier it is to separate by using the distillation column. The vapor with high volatility will flow up the column and the liquid with low volatility and dissolved flows counter currently down the column. The top part of the column is the rectifying section called distillate and the bottom part of the column is stripping section called the bottom. The reflux ratio is the ratio of the reflux going back down the distillation column and the amount of reflux collected in the distillate, and the formula is stated below.

$$R = ( flow returned as reflux ) / ( flow of top product taken off ) \qquad Equation 1$$

The optimum reflux ratio is between the minimum specified separation and the total reflux. By having a higher reflux ratio, the number of stages required reduces which would decrease the capital cost. The feed point location would affect the number of stages required and the amount of product produced. The optimum feed point is normally the midpoint of the total stages that separate the liquid and vapor in the column. In some cases, the feed point for azeotrope distillation is the first stage of the column due to stripping the water towards the bottom.

Based on the close boiling points and volatility of the oxygenates, it is difficult to separate the components due to azeotropes. However, due to the interaction of ethanol and water, the multiple azeotrope issue was avoided, and simple column distillation was sufficient to obtain a pure product. The boiling points and volatility of the components are represented in Tab ‘Boiling points’ in the Material Balance sheet.

### Economic Evaluation

In one embodiment, the integrated process for transforming coals into ethanol comprises chemical looping to transform coals into syngas with an H₂/CO ratio of 2. The thus generated syngas (with 400 H₂S) is supplied to the reactor to produce almost exclusively EtOH while the unreacted syngas is recycled back to the reactor to further transformed into ethanol. This mode of operation at the selected operating conditions of the main reactors is basically transforming all syngas into ethanol. The acid gases that are produced from the chemical looping process and the small amount of H₂S that is coming out of the main reactor (H₂S is fed into the main reactor to ensure stability and promote the almost exclusive production of EtOH) is being fed to the Selexol for purification. The sulfur is also transformed into H₂SO₄ that adds to the total revenue of the process. The process can operate 24 hours a day year-round, excluding holidays and scheduled and unscheduled downtime, for about 8,000 operating hours a year. Coal can be fed into the process at 3,000 tons per day.

As an example of the economic considerations, a detailed list of the Capital Cost for the Chemical Looping (CL), the Acid Gas purification (PURIFY), and the main reactor to transform Syngas into Oxygenates (OXY) are presented in Table 3. The total capital investment for the integrated process is $849.5 million. The revenue and the operating cost for three individual processes of the integrated process that will transform coals into ethanol is presented below in Table 17. Total cost excluding depreciation (depreciation for the integrated process is $47.1 million/year) is $322.4 million/year. The total revenue from the integrated process which includes the manufacturing and selling of H₂SO₄ from H₂S reaches $476.6 million/year. This results in a net profit of $154,200,000/year for processing 3,000 tons/day of coal. The proposed process of transforming coals into ethanol seems to be a viable process.

### EXAMPLES

### Example 1—Synthesizing Ethanol from Syngas

Incoming syngas was sent from the chemical looping of coals process. The syngas is composed of hydrogen gas and carbon monoxide at a 2:1 ratio and hydrogen sulfide at 400 ppm to suppress side reactions. The syngas is then fed into the main reactor where primarily ethanol and water are produced. The reactor exit stream is then fed into a heat exchanger to separate the unreacted non-condensable materials (hydrogen, carbon monoxide and hydrogen sulfide) from the condensable materials (water, ethanol, and other trace oxygenate). The non-condensable stream is recycled back into the reactor with a small purge stream of primarily hydrogen leaving the system to compensate for the limiting reactant carbon monoxide. The condensable stream is then sent to the separations section of the process where the stream is first brought to atmospheric pressure by feeding the stream through an expander. The stream is then cooled through a heat exchanger to 50° C. and fed to the first of two distillation towers.

The first tower separated a 97% by weight water stream out the bottom of the column and a 96% by weight ethanol stream exiting the top of the column. The top stream was then sent to a second column, which further purified the ethanol stream to 98% by weight with a small waste stream from the top of the column being fed to a mixer that mixed the water stream from the first column.

### Example 2—Effect of H₂S on the Catalytic Performance

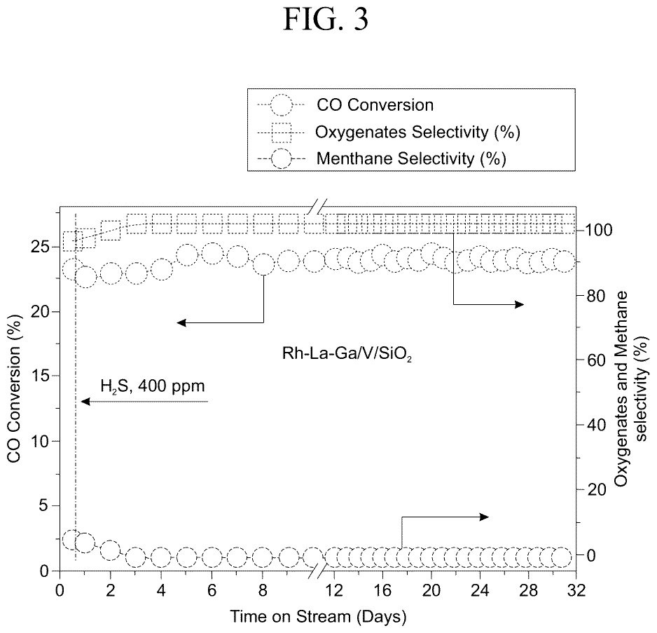

The effect of a sulfur stream (400 ppm H₂S) on the oxygenates distribution for the 1.5Rh-1.5La-1Ga/1.8V/SiO₂ catalyst during 31 days of the reaction was determined. The selectivity trends of all oxygenate products over the 1.5Rh-1.5La-1Ga/1.8V/SiO₂ catalyst during the time on stream study are displayed in FIG. 3 and Table 18. As can be observed, the 1.5Rh-1.5La-1Ga/1.8V/SiO₂ catalyst exhibited the ˜29% and ˜50% selectivity towards the ethanol (EtOH) and formaldehyde (HCHO) formation, respectively, at the initial stage of the reaction without H₂S stream. Surprisingly, the selectivity of HCHO decreased after the introduction of 400 ppm H₂S during 5 days on stream. At the same time, the decrease in selectivity towards HCHO over the catalyst is accompanied by an increase in selectivity of EtOH (FIG. 3 and Table 18). The selectivities of HCHO and EtOH became stable at around 2% and 96%, respectively, after about 5 days of the reaction. This finding is particularly interesting because ethanol is an industrially more valuable product than that of formaldehyde.

Moreover, the selectivity towards C₂₊ oxygenates and methanol decreased when sulfur was fed into the reaction stream. Especially, the selectivity of C₂₊ oxygenates dramatically dropped from ˜12% to ˜1% during the time on stream study in presence of 400 ppm H₂S. The decrease in the selectivities of C₂₊ oxygenates and methanol is also accompanied by an increase in ethanol selectivity. Hence, the presence of sulfur in the reaction stream was very beneficial for the selective conversion of syngas into ethanol by decreasing the selectivity towards other oxygenates. This behavior is much desired because it will make the process design significantly simpler and will require lower capital investment.

### Example 3—Effect of Different Sulfur Concentrations

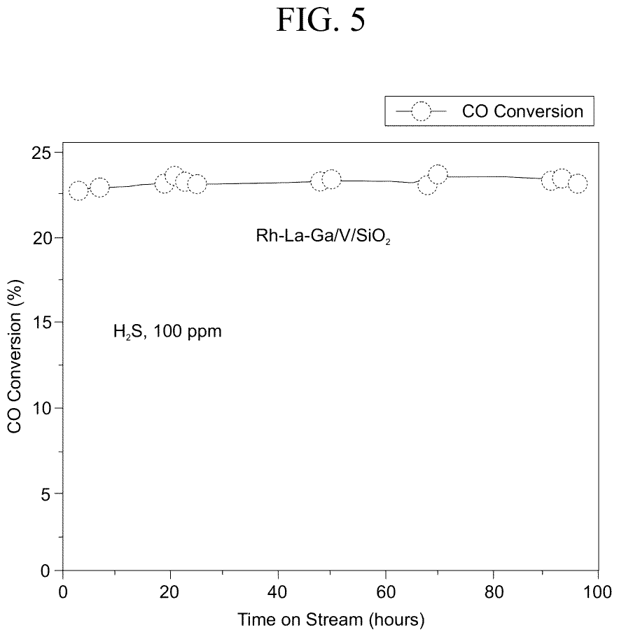

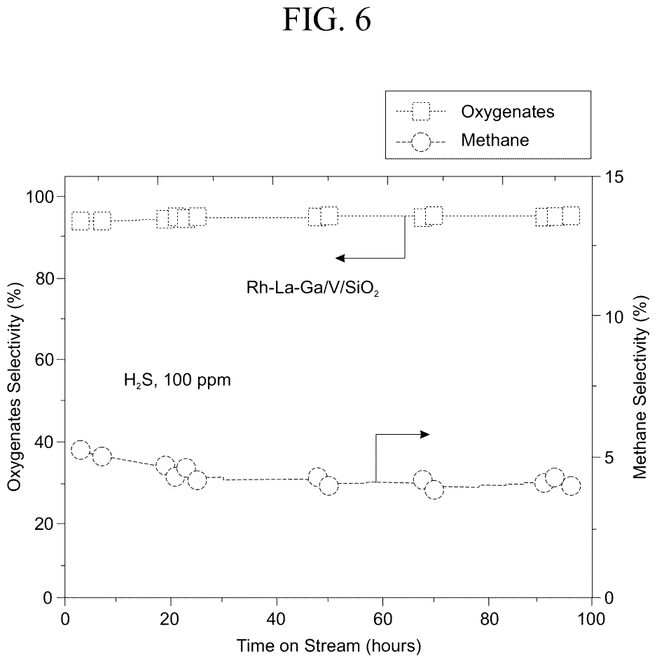

The effect of sulfur concentration ranging from 100 to 1000 ppm on the performance of Rh—La—Ga/V/SiO₂ catalyst in syngas to oxygenates reaction at 260° C. under high pressure (P=350 psig) was investigated. FIG. 5 and Table 19 show the CO conversion over the catalyst in presence of 100 ppm H₂S during 96 h of the reaction. As can be observed, the CO conversion over the catalyst was not affected by the presence of 100 ppm H₂S in the feed gas. As shown in FIG. 6 and Table 19, the catalyst showed ˜4.5% and ˜95.5% selectivity towards methane and oxygenates products, respectively, even in presence of 100 ppm H₂S in the reaction stream. Thus, it can be presumed that the 100 ppm H₂S in the syngas could not be sufficient to inhibit the methane formation to zero.

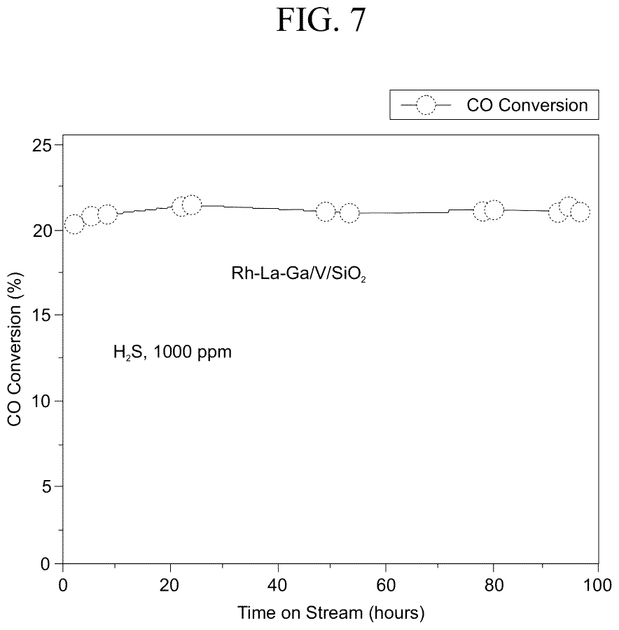

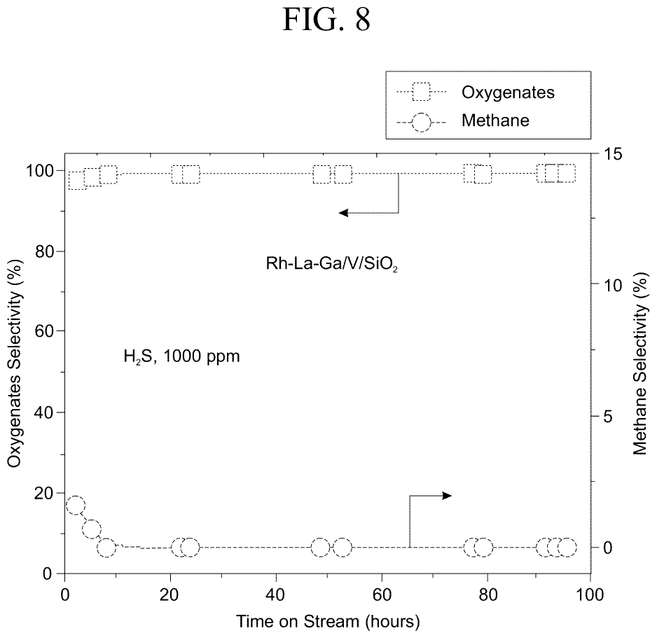

FIGS. 7 and 8 present the catalytic performance of Rh—La—Ga/V/SiO₂ for syngas to oxygenates reaction in presence of 1000 ppm H₂S for 4 days (Table 20). The CO conversion decreased (˜21.1%) upon the addition of 1000 ppm H₂S into the reaction stream (FIG. 7 and Table 20) compared to that CO conversion (˜23.8%) obtained at 400 ppm sulfur. Besides, the 1000 ppm H₂S in the feed leads to increased oxygenates selectivity to 100% within 8 hours of the reaction by decreasing the methane selectivity to zero (FIG. 8 and Table 20). From these findings, it can be concluded that the presence of ≥400 ppm H₂S in the feed gas is helpful to completely suppress the methane formation by maintaining stable CO conversion. Moreover, the cofeeding of H₂S is extremely beneficial because it suppresses the selectivities of CH₄, HCHO and higher alcohols to very small amounts while promotes the almost exclusive production of EtOH. This behavior is of great importance for the effective and economic design of the process to transform coal generated syngas to EtOH.

### Example 4—Integrated Process

Raw bituminous coal is fed into a grinder, U-100, at 3,000 tons per day, or 125 tons per hour. Once the coal has been crushed to approximately 100 mesh and preheated in E-110 using the partially cooled syngas, the stream enters the next production area, the reactions phase. The coal is mixed with a fresh Fe₂O₃ feed, pressurized to 10 bar on entrance to the V-225 Reaction Pressure Vessel, and enters the R-200 Reduction Reactor along with a recycled stream of Fe₂O₃ and steam. This reaction produces the product, syngas, and byproducts including fly ash, deactivated catalyst, and solid FeO. The syngas is passed through two heat exchangers and a decanter to remove the water before being sent on to the next processing step. The water is recycled and reboiled before being fed back into R-200. The solid FeO is hoisted with a screw conveyer into a R-250 Combustion Reactor and mixed with air, where the FeO is oxidized to form Fe₂O₃ and depleted air. The regenerated Fe₂O₃ is recycled back to R-200. An equipment list can be seen in Table 9.

The good heat transfer properties of fluidized beds are taken advantage of to minimize the amount of preheating done for solid reactants. R-200 runs at 1590° F. and therefore its effluent streams leave at that temperature. R-250 runs at 2160° F. for the same effect. For instance, the preheated coal-fresh catalyst mixture enters R-200 at 940° F. while the recycled catalyst enters at 2160° F.

All product streams are cooled to 90° F. before leaving battery limits with their energy recovered into the process. This allows all external heat for the process to be supplied by H-205 directly to R-200, saving on cost and complexity.

In response to changes in coal composition, the process flows can be adjusted to compensate. For instance, when oxygen rich coal is fed to the process, the flow of oxygen carrying catalyst will be reduced or when hydrogen rich is fed, the flow of water will be reduced. In this way, the mass balance will be maintained.

For this design, ChemCAD was used primarily for PFD drawings, to model the condensation of water from syngas (see Table 10) and for the energy integration of the syngas/steam cross exchanger and air/spent air exchanger.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

### TABLES

TABLE 1
Assumed Coal Composition from U.S. Geological Survey

| Component | Min % by weight | Max % by weight |
|---|---|---|
| C | 64.0 | 76.0 |
| H | 4.9 | 5.3 |
| O | 4.9 | 8.6 |
| N | 1.3 | 2.0 |
| S | 2.4 | 6.8 |
| Ash | 5.5 | 18.0 |

TABLE 2
Feed Streams

| Stream | Flowrate (ton/hr) |
|---|---|
| Ohio bituminous coal | 125 |
| Fe₂O₃ catalyst, raw | 7-10 |
| Process Water | 120 |
| Air | 380-520 |

TABLE 3
Classification of Coals by Rank

| | Fixed carbon limits (dry, | | Volatile matter limits (dry, | |
|---|---|---|---|---|
| | mineral-matter-free basis), % | | mineral-matter-free basis), % | |
| | Equal or | Less | Greater | Equal or |
| Class/group | greater than | than | than | less than |
| Low-volatile bituminous coal | 78 | 86 | 14 | 22 |
| Medium-volatile bituminous coal | 69 | 78 | 22 | 31 |

| | |
|---|---|
| Specific Heat KJ/(kgK) | 1.0-1.1 |
| Specific Heat Btu/(lb ° F.) | 0.24-0.25 |
| Bulk Density kg/m³ | 670-910 |
| Bulk Density lb/ft³ | 42-57 |

TABLE 4
Flowrates of Dry Syngas Components

| Component | % by weight | Minimum tons/hr | Maximum tons/hr |
|---|---|---|---|
| H₂ | 0.07 | 17.5 | 21 |
| CO | 0.50 | 125 | 150 |
| CO₂ | 0.38 | 95 | 114 |
| CH₄ | 0.005 | 1.25 | 1.5 |
| N₂ | 0.006-0.008 | 1.5 | 2.4 |
| H₂S | 0.011-0.036 | 2.75 | 10.8 |
| Total | — | 240 | 280 |

TABLE 5
Flowrates by Product and Byproduct Streams

| Component | Minimum tons/hr | Maximum tons/hr |
|---|---|---|
| Syngas | 240 | 280 |
| Fly Ash | 10 | 40 |
| Deactivated catalyst | 7 | 10 |
| Spent Air | 340 | 470 |

TABLE 6
Utility Streams

| Stream | Utility | Requirement | |
|---|---|---|---|
| H-205 Reactor Heater | Coal | 25 | tons/hr |
| U-100⁷ and Conveyers | Electricity | 12MM | kWh |

TABLE 7
Cost of Coal with Delivery Costs

| | | | | $MM/year |
|---|---|---|---|---|
| Avg. Bituminous Ohio | Avg. $/ton | Total | | Coal + |
| Coal Price $42/ton in | Delivery | $/ton + | $MM/ | Delivery |
| 2018⁹ | Costs¹⁰ | Delivery | day | Cost |
| Rail | 21 | 63 | 0.19 | 63 |
| Waterways | 4 | 46 | 0.14 | 47 |
| Trucking | 10 | 52 | 0.16 | 52 |

TABLE 8
Comparison of Fuel Heating Sources

| Solid or Liquid | $/ | MMBtu/ | MMBtu/ | Tons/ | Heating | Heating |
|---|---|---|---|---|---|---|
| Fuel Heating Source | ton | ton | ton | hr | Cost $/hr | Cost $MM/yr |
| Coal | 46 | 1.2*10⁻² | 24 | 52 | 2,500 | 20 |
| Formaldehyde | 80 | 8.2*10⁻⁶ | 0.01634 | 77,100 | 6.0*10⁶ | 49,400 |

| Gas Fuel | $/MM | MMBtu/ | MMBtu/ | Tons/ | Heating | Heating |
|---|---|---|---|---|---|---|
| Heating Source | SCF | SCF | ton | hr | Cost $/hr | Cost $MM/yr |
| Natural Gas | 3.35 | 9.8*10⁻⁴ | 41 | 31 | 4,300 | 34 |
| Syngas | 2,500 | 1.2*10⁻⁴ | 0.48 | 2624 | 2.6*10¹⁰ | 209,889,608 |

TABLE 9
Equipment List for the Chemical Looping

Tag

| Number | Equipment Description | Sizing | |
|---|---|---|---|
| U-100 | Coal Hammer Mill 30 mm −> 100 mesh | 125 | tons/hr |
| | Carbon Steel | | |
| E-110 | Coal Preheater Fixed Head Shell and | 10,000 | ft² |
| | Tube (sizing an estimate due to solids/ | | |
| | gas exchange) Carbon Steel | | |
| E-120 | Fresh Air Heater/Depleted Air Cooler, | 10,000 | ft² each |
| A-H | Fixed Head Shell and Tube, 8 units | | |
| | (76,000 ft² total) 304SS | | |
| R-200 | Coal/Fe₂O₃ Reduction Reactor 24″ | 44,000 | ft³ |
| | thick Refractory Brick with 2″ Alumina | | |
| | Liner | | |
| H-205 | Reduction Reactor Heater 304SS, coal | 601 | MMBtu/hr |
| | fired | | |
| V-225 | Pressure Vessel to contain reactors and | 113,000 | ft³ |
| | hot processing steps 304SS | | |
| R-250 | FeO Combustion Reactor 24″ thick | 23,000 | ft³ |
| | Refractory Brick with 2″ Alumina Liner | | |
| F-300 | Syngas/Fly Ash Separation Cyclone | 136,000 | ft³/min |
| | 304SS | | |
| F-310 | Fe₂O₃/Spent Air Separation Cyclone | 104,000 | ft³/min |
| | 304SS | | |
| E-320 | Water Condenser, Fixed Head Shell and | 10,000 | ft³ each |
| A-G | Tube, 7 units (68,000 ft² total) 304SS | | |
| V-340 | Water Decanter Carbon Steel | 80,000 | gal water/hr |

TABLE 10
Wet vs. Dry Syngas Conditions

| | Wet Syngas | Dry Syngas |
|---|---|---|
| Flow Rate (tons/hr) | 650-750 | 240-280 |
| H₂ (wt %) | 3% | 7% |
| CO (wt %) | 19% | 51% |
| CO₂ (wt %) | 14% | 39% |
| H₂O (wt %) | 63% | 1% |
| CH₄ (wt %) | 0.2% | 0.5% |
| N₂ (wt %) | 0.2% | 0.5% |
| H₂S (wt %) | 0.4-1.4% | 1.1-3.6% |
| Temperature (° F.) | 1590 | 90 |
| Pressure (bar) | 10 | 10 |
| Specific Gravity (lb/ft3) | 0.112 | 0.3809 |
| Viscosity (cP) | 0.0429 | 0.0163 |

TABLE 11
Feedstock Specifications for Syngas Selexol Process

| Property | Syngas Feed | Initial/Recycling Selexol |
|---|---|---|
| Temperature (° F.) | 85 | 90.7046 |
| Pressure (psig) | 986 | 1,000 |
| Mass Flow (lb/hr) | 494,400 | 1,000,000 |
| Molar Flow (lbmol/hr) | 31,889.86 | 3,850.498 |
| Vapor Fraction | 0.9979693 | 0 |
| Enthalpy (MMBtu/hr) | −1,169.977 | −169.3498 |

TABLE 12
Feedstock Specifications for Oxygenates Effluent Selexol Process

| | Oxygenates | Initial/Recycling | Scrubbing |
|---|---|---|---|
| Property | Effluent | Selexol | Water |
| Temperature (° F.) | 122 | 107 | 100 |
| \Pressure (psig) | 330 | 986 | 50 |
| Mass Flow (lb/hr) | 18,286.52 | 350,000.00 | 69977 |
| Molar Flow (lbmol/hr) | 5,194.929 | 1,314.159 | 3,884.374 |
| Vapor Fraction | 1 | 0 | 0 |
| Enthalpy (MMBtu/hr) | −12.8682 | −50.19495 | −476.0257 |

TABLE 13
Feedstock Specifications for Wet Sulfuric Acid Process

| | Combined Acid | | |
|---|---|---|---|
| | Gas Streams | | |
| | from Selexol | Combustion | Lime |
| Property | Processes | Air | Water |
| Temperature (° F.) | 779.649 | 72 | 68 |
| Pressure (psig) | 45.3 | 0 | −0.2 |
| Mass Flow (lb/hr) | 161,024.2 | 154,708 | 11,983.56 |
| Molar Flow (lbmol/ | 5,545.139 | 5,362.337 | 248.7635 |
| hr) | | | |
| Vapor Fraction | 1 | 1 | 0 |
| Enthalpy (MMBtu/ | 22.76197 | −0.1868011 | −62.70133 |
| hr) | | | |

TABLE 14
Key Process Equipment for the separation of the Acid gases

| | Tag | | Sizing |
|---|---|---|---|
| Process Unit | Number | Description | Parameter |
| Syngas Selexol | CL-124 | H₂S Absorber | 10 stages |
| Syngas Selexol | CL-131 | H₂S Concentrator | 11 stages |
| Syngas Selexol | CL-132 | Selexol ™ Regenerator | 7 stages |
| Syngas Selexol | CL-109 | CO₂ Absorber | 20 stages |
| Syngas Selexol | CL-104 | CO₂ Stripper | 5 stages |
| Oxy. Effluent | CL-216 | H₂O/HCHO Scrubber | 10 stages |
| Selexol | | | |
| Oxy. Effluent | CL-207 | H₂S Absorber | 10 stages |
| Selexol | | | |
| Oxy. Effluent | CL-214 | H₂S/Selexol Stripper | 10 stages |
| Selexol | | | |
| Wet Sulfuric Acid | R-302 | H₂S Combustion Reactor | 48 ft diameter |
| Wet Sulfuric Acid | R-304 | SO₂ Catalytic Reactor | 48 ft diameter |
| Wet Sulfuric Acid | R-306 | SO₂ Catalytic Reactor | 48 ft diameter |
| Wet Sulfuric Acid | R-307 | SO₂ Catalytic Reactor | 48 ft diameter |
| Wet Sulfuric Acid | R-308 | Gas Cooler | 48 ft diameter |
| Wet Sulfuric Acid | CL-313 | SO₂ Scrubber | 2 stages |
| Wet Sulfuric Acid | CL-314 | Wet Electrostatic | Outside Shell: 9.5 |
| | | Precipitator | ft × 11 ft |

TABLE 15
Equipment List for the Syngas to Ethanol project

| | TAG | PROCESS | |
|---|---|---|---|
| EQUIPMENT NAME | NUMBER | SERVICE | SIZE(FT²) |
| SYNGAS PUMP | P-100 | PUMP | — |
| SYNGAS REACTOR | R-105 | REACTOR | 1500 |
| HEAT EXCHANGER | E-110 | HEAT EXCHANGER | 87363.35 |
| COMPRESSOR | C-115 | COMPRESSOR | — |
| HEAT EXCHANGER | E-120 | HEAT EXCHANGER | 50 |
| ETHANOL-WATER SEPARATION | CL-125 | SEPARATION | — |
| COLUMN | | COLUMN | |
| ETHANOL-WATER HEAT EXCHANGER | E-130 | HEAT EXCHANGER | 22017 |
| ETHANOL-WATER CONDENSER | E-135 | CONDENSER | 10146.61 |
| PURE ETHANOL SEPARATION | CL-140 | SEPARATION | — |
| COLUMN | | COLUMN | |
| PURE ETHANOL HEAT EXCHANGER | E-145 | HEAT EXCHANGER | 22017 |
| PURE ETHANOL CONDENSER | E-150 | CONDENSER | 10146.61 |
| REACTANTS RECYCLE PUMP | P-155 | PUMP | — |
| REACTANTS MIXER | A-220 | MIXER | — |
| WATER MIXER | A-225 | MIXER | — |
| TOWER WATER SUPPLY | TWS-300 | TOWER WATER | — |
| TOWER WATER RETURN | TWR-305 | TOWER WATER | — |
| TOWER WATER SUPPLY | TWS-310 | TOWER WATER | — |
| TOWER WATER RETURN | TWR-315 | TOWER WATER | — |

TABLE 16
Capital Cost Estimate for each individual Process

| CAPITAL INVESTMENT | CL | PURIFY | OXY | Total |
|---|---|---|---|---|
| Purchased Equipment¹ (CE Index = 567) | $35.7 | $42.1 | $6.1 | $83.9 |
| Bare Module Cost² (CE Index = 640) | $93.7 | $126.7 | $15.3 | $235.6 |
| Initial Materials | | | | |
| Iron Oxide | | | | $68.0 |
| Selexol | | | | $1.8 |
| Catalyst | | | | $2.5 |
| Total Bare Module Cost (Including Initial Materials) | | | | $307.9 |
| Site Preparation (15% of Total Bare Module Cost) | | | | $46.2 |
| Service Facilities (10% of Total Bare Module Cost) | | | | $30.8 |
| Allocated Capital | | | | |
| Tower Water | | | | $2.3 |
| Chill Water | | | | $6.2 |
| Refrigeration | | | | $0.5 |
| 90# Steam | | | | $13.1 |
| 1200# Steam | | | | $32.2 |
| Electricity | | | | $1.4 |
| Total Allocated Capital Costs | | | | $55.8 |
| Direct Permanent Investment (DPI) | | | | $440.7 |
| Contingency (35% of DPI) | | | | $154.2 |
| Contractors' Fee (3% of DPI) | | | | $13.2 |
| Total Depreciable Capital (TDC) | | | | $608.1 |
| Land (2% of TDC) | | | | $12.2 |
| Royalties (2% of TDC) | | | | $12.2 |
| Startup (25% of TDC) | | | | $152.0 |
| TOTAL PERMANENT INVESTMENT | | | | $784.5 |
| Working Capital | | | | |
| Cash Reserves (1 month of COM) | | | | $27.0 |
| Inventory (1 week of Sales) | | | | $9.1 |
| Accounts Receivable (1 month of Sales) | | | | $39.2 |
| Less Accounts Payable (1 month of Raw Materials) | | | | −$10.9 |
| Total Working Capital | | | | $65.0 |
| TOTAL CAPITAL INVESTMENT | | | | $849.5 |

Results reported in millions of mid-2020 US dollars unless otherwise noted.

TABLE 17
Revenue and Operating Cost Estimate for each individual Process

| | CL | PURIFY | OXY | Total |
|---|---|---|---|---|
| Revenue | | | | $476.6 |
| Ash | $3.6 | | | |
| Sulfuric Acid | | $8.3 | | |
| Ethanol³ | | | $464.7 | |
| Raw Materials and Catalysts | | | | $133.2 |
| Coal for Looping | $52.0 | | | |
| Coal for Heating | $10.4 | | | |
| Iron Oxide | $68.0 | | | |
| Water | $0.3 | ($0.1) | ($0.1) | |
| Make-Up Selexol | | $0.2 | | |
| Catalyst | | | $2.48 | |
| Utilities | | | | $44.0 |
| Tower Water | | $6.4 | $1.3 | |
| Chill Water | | $0.6 | | |
| Refrigeration | | $0.1 | | |
| 90# Steam | | $0.5 | $5.4 | |
| 1200# Steam | | $29.6 | | |
| Electricity | $0.2 | <$0.1 | <$0.1 | |
| Operations | | | | $8.0 |
| Maintenance | | | | $69.9 |
| Operating Overhead | | | | $10.1 |
| Property Taxes and Insurance | | | | $12.2 |
| COST OF MANUFACTURE | | | | $277.3 |
| excluding Depreciation | | | | |
| GENERAL EXPENSES | | | | $55.1 |
| TOTAL COST excluding | | | | $322.4 |
| Depreciation | | | | |

Results reported in millions of mid-2020 US dollars unless otherwise noted.

TABLE 18
CO conversion, and the selectivity of various oxygenates and methane in the syngas
to oxygenates reaction over Rh—La—Ga/V/SiO₂ at 260° C. for 31 days; Reaction conditions:
H₂S = 400 ppm, P = 350 psig, H₂/CO = 2, S.V. = 6,000 sccm gcat⁻¹h⁻¹.

| | | | | | | | C2+ |
|---|---|---|---|---|---|---|---|
| Time on | CO | Methane | Oxygenates | HCHO | MeOH | EtOH | Oxygenates |
| Stream | Conversion | Selectivity | Selectivity | Selectivity | Selectivity | Selectivity | Selectivity |
| (Days) | (%) | (%) | (%) | (%) | (%) | (%) | (%) |
| 0.5 | 23.2 | 5.4 | 94.6 | 50.4 | 3.3 | 28.9 | 12 |
| 1 | 22.6 | 4.4 | 95.6 | 45.2 | 2.5 | 41 | 6.9 |
| 2 | 22.9 | 2.1 | 97.9 | 30.1 | 1.2 | 63.8 | 2.8 |
| 3 | 22.9 | 0 | 100 | 16.6 | 1.7 | 80 | 1.7 |
| 4 | 23.2 | 0 | 100 | 7.4 | 1.4 | 90 | 1.2 |
| 5 | 24.3 | 0 | 100 | 1.5 | 1.1 | 96.4 | 1 |
| 6 | 24.4 | 0 | 100 | 2 | 1.2 | 96 | 0.8 |
| 7 | 24.2 | 0 | 100 | 1.9 | 1.4 | 95.4 | 1.3 |
| 8 | 23.6 | 0 | 100 | 2 | 1.3 | 95.6 | 1.1 |
| 9 | 23.9 | 0 | 100 | 2.3 | 1.4 | 95 | 1.3 |
| 10 | 23.8 | 0 | 100 | 2.4 | 1.6 | 94.7 | 1.3 |
| 12 | 24 | 0 | 100 | 1.5 | 0.9 | 96.9 | 0.7 |
| 13 | 24.1 | 0 | 100 | 2.4 | 1.8 | 94.4 | 1.4 |
| 14 | 23.7 | 0 | 100 | 1.6 | 1.2 | 96.2 | 1 |
| 15 | 23.9 | 0 | 100 | 1.7 | 1 | 96.4 | 0.9 |
| 16 | 24.3 | 0 | 100 | 1.5 | 1.1 | 96.4 | 1 |
| 17 | 23.8 | 0 | 100 | 1.6 | 0.9 | 96.7 | 0.8 |
| 18 | 24.1 | 0 | 100 | 1.8 | 1.2 | 96 | 1 |
| 19 | 23.9 | 0 | 100 | 1.7 | 1 | 96.5 | 0.8 |
| 20 | 24.4 | 0 | 100 | 1.1 | 0.6 | 97.7 | 0.6 |
| 21 | 24.1 | 0 | 100 | 2.2 | 1.4 | 95.2 | 1.2 |
| 22 | 23.7 | 0 | 100 | 2 | 1.3 | 95.6 | 1.1 |
| 23 | 23.9 | 0 | 100 | 1.9 | 1.2 | 95.9 | 1 |
| 24 | 24.2 | 0 | 100 | 1.3 | 0.8 | 97.3 | 0.6 |
| 25 | 23.8 | 0 | 100 | 2 | 1.3 | 95.8 | 0.9 |
| 26 | 23.9 | 0 | 100 | 1.6 | 1.1 | 96.4 | 0.9 |
| 27 | 24.1 | 0 | 100 | 1.7 | 1 | 96.5 | 0.8 |
| 28 | 23.7 | 0 | 100 | 2 | 1.1 | 96 | 0.9 |
| 29 | 23.8 | 0 | 100 | 2.5 | 1.6 | 94.5 | 1.4 |
| 30 | 24 | 0 | 100 | 2.7 | 1.7 | 94.1 | 1.5 |
| 31 | 23.8 | 0 | 100 | 1.6 | 1.2 | 96.5 | 0.7 |

TABLE 19
CO conversion and the selectivity towards oxygenates and methane
in the syngas to oxygenates reaction over Rh—La—Ga/V/SiO2
at 260° C. for 4 days; Reaction conditions: H2S =
100 ppm, P = 350 psig, H2/CO = 2, S.V. = 6,000 sccm gcat−1h−1.

| Time on | CO | Methane | Oxygenates |
|---|---|---|---|
| Stream (Days) | Conversion (%) | Selectivity (%) | Selectivity (%) |
| 3 | 22.7 | 5.3 | 94.7 |
| 7 | 22.9 | 5.1 | 94.9 |
| 19 | 23.1 | 4.8 | 95.2 |
| 21 | 23.6 | 4.4 | 95.6 |
| 23 | 23.2 | 4.7 | 95.3 |
| 25 | 23.1 | 4.3 | 95.7 |
| 48 | 23.2 | 4.4 | 95.6 |
| 50 | 23.4 | 4.1 | 95.9 |
| 68 | 23 | 4.3 | 95.7 |
| 70 | 23.7 | 4 | 96.0 |
| 91 | 23.3 | 4.2 | 95.8 |
| 93 | 23.4 | 4.4 | 95.6 |
| 96 | 23.1 | 4.1 | 95.9 |

TABLE 20
CO conversion and the selectivity towards oxygenates and methane
in the syngas to oxygenates reaction over Rh—La—Ga/V/SiO2
at 260° C. for 4 days; Reaction conditions: H2S =
1,000 ppm, P = 350 psig, H2/CO = 2, S.V. = 6,000 sccm gcat−1h−1.

| Time on | CO | Methane | Oxygenates |
|---|---|---|---|
| Stream (Days) | Conversion (%) | Selectivity (%) | Selectivity (%) |
| 2 | 20.4 | 1.5 | 98.5 |
| 5 | 20.9 | 0.6 | 99.4 |
| 8 | 21 | 0 | 100 |
| 22 | 21.5 | 0 | 100 |
| 24 | 21.6 | 0 | 100 |
| 49 | 21.2 | 0 | 100 |
| 53 | 21.1 | 0 | 100 |
| 78 | 21.2 | 0 | 100 |
| 80 | 21.3 | 0 | 100 |
| 92 | 21.1 | 0 | 100 |
| 94 | 21.5 | 0 | 100 |

What is claimed is:

1. A process for producing oxygenates from syngas, the process comprising the steps of
a) mixing syngas with hydrogen sulfide, forming a syngas mixture;
b) contacting the syngas mixture with a catalyst composition in a first reactor to obtain a first product stream comprising ethanol, water, hydrogen, carbon monoxide and hydrogen sulfide;
c) splitting the first product stream into a non-condensable material stream comprising hydrogen, carbon monoxide and hydrogen sulfide and a condensable material stream comprising water and ethanol;
d) directing the non-condensable material stream back into the first reactor; and
e) directing the condensable material stream to a first distillation column, producing a second product stream comprising a majority of water and a third product stream comprising a majority of ethanol;
wherein the catalyst composition comprises rhodium, lanthanum, gallium, vanadium and a support.

2. The process of claim 1 wherein the support for the catalyst composition is selected from the group comprising SiO₂, zeolites, silicalites, mesoporous silicas, alumina, aluminophosphates, activated carbons, and carbon nanotubes.

3. The process of claim 1 wherein the support for the catalyst composition is SiO₂.

4. The process of claim 1 wherein the catalyst composition comprises 1.5Rh-1.5La-1Ga/1.8V/SiO₂.

5. The process of claim 1 wherein the hydrogen sulfide is present in the syngas mixture at a level of 400 ppm or greater.

6. The process of claim 1 wherein the hydrogen sulfide is present in the syngas mixture at a level of about 400 ppm.

7. The process of claim 1 wherein the third product stream is directed to a second distillation column, producing a fourth product stream comprising a majority of water and a fifth product stream comprising a majority of ethanol.

8. The process of claim 1 wherein the third product stream comprises at least about 90 weight percent ethanol.

9. The process of claim 1 wherein the third product stream comprises at least about 95 weight percent ethanol.

10. The process of claim 7 wherein the fifth product stream comprises at least about 90 weight percent ethanol.

11. The process of claim 7 wherein the fifth product stream comprises at least about 95 weight percent ethanol.

12. The process of claim 1 wherein the syngas has a hydrogen (H₂) to carbon monoxide (CO) molar ratio of about 1-4.

13. The process of claim 1 wherein the syngas has a hydrogen (H₂) to carbon monoxide (CO) molar ratio of about 2.

14. The process of claim 1 wherein the first reactor is operated at a temperature from about 150° C. to about 350° C., a space velocity of about 5000-7000 sccm gcat⁻¹h⁻¹ and a pressure of between atmospheric and about 700 psig.

15. The process of claim 1 wherein the first reactor is operated at a temperature of about 260° C.

16. The process of claim 1 wherein the first reactor is operated at a pressure of about 350 psig.

17. The process of claim 1 wherein the first reactor is operated at a space velocity of about 6000 sccm gcat⁻¹h⁻¹.

18. The process of claim 1 wherein the first product stream is split into a non-condensable material stream and a condensable material stream using a heat exchanger.

19. The process of claim 1 wherein the syngas is sourced from coal.

20. The process of claim 19 wherein the syngas is treated to remove acid gases using a physical solvent that is a mixture of dimethyl ethers of polyethylene glycols prior to mixing the syngas with hydrogen sulfide.
